(19) United States
(12) Patent Application Publication
(10) Pub. No.: US 2020/0009351 A1
(43) Pub. Date: Jan. 9, 2020

(54) Carotid Stent Incorporating Arch Fulcrum Catheters and Flow Reversal

(71) Applicant: Daniel Ezra Walzman, Bergenfield, NJ (US)

(72) Inventor: Daniel Ezra Walzman, Bergenfield, NJ (US)

(21) Appl. No.: 16/575,302

(22) Filed: Sep. 18, 2019

Related U.S. Application Data
(63) Continuation-in-part of application No. 16/501,577, filed on May 2, 2019.
Application No. 16/501,577 is a continuation-in-part of application No. 16/290,923, filed on Mar. 3, 2019.
Application No. 16/290,923 is a continuation-in-part of application No. 15/932,775, filed on Apr. 23, 2018.
Application No. 15/932,775 is a continuation-in-part of application No. 15/250,693, filed on Aug. 29, 2016.
Application No. 15/250,693 is a continuation of application No. 15/158,341, filed on May 18, 2016, now abandoned.

(51) Int. Cl. A61M 25/00; A61B 5/00; A61F 2/856; A61F 2/954; A61M 25/10

(52) U.S. Cl. CPC A61M 25/0041; A61B 5/6852; A61F 2/856; A61F 2002/9528; A61M 25/0052; A61M 25/10; A61F 2/954

(57) ABSTRACT

A medical device capable of treating a carotid blockage or narrowing with multiple therapeutic devices simultaneously, comprising an arch-fulcrum support catheter tube having at least one lumen from a proximal port to a distal end hole for delivering additional medical devices, a second lumen to inflate at least one distal, circumferential balloon or swellable, circumferential hydrogel, capable of temporary occlusion of native flow in a vessel near its distal end hole upon inflation, a wire for delivery of additional devices, a second balloon for angioplasty, at least one stent. Flow is allowed through said proximal port, flow across said vascular narrowing is reversed, thereby allowing debris released during angioplasty and stenting to flow out of said catheter, avoiding potential thromboembolic complications in the distal vasculature, and associated embodiments.

### CROSS-REFERENCE(S)

This is a continuation-in-part (CIP) application claiming the benefit of priority to U.S. Non-Provisional application Ser. No. 16/501,577 filed May 2, 2019 which is a CIP application claiming the benefit of U.S. Non-Provisional application Ser. No. 16/290,923 filed Mar. 3, 2019; which is a CIP application claiming the benefit of Ser. No. 15/932,775 filed Apr. 23, 2018, which is a CIP claiming the benefit of priority to Ser. No. 15/250,693 filed Aug. 29, 2016, which in turn claims priority to application Ser. No. 15/158,341 filed on May 18, 2016, the entire contents of which are incorporated by reference and further to the following U.S. nonprovisional patent appl. Ser. Nos. 16/501,806, filed Jun. 10, 2019 (10 Jun. 2019); Ser. No. 16/501,592, filed May 2, 2019 (2 May 2019); Ser. No. 16/214,130, filed Dec. 9, 2018 (9 Dec. 2018); Ser. No. 16/151,335, filed Oct. 3, 2018 (3 Oct. 2018); Ser. No. 16/125,691, filed Sep. 8, 2018 (8 Sep. 2018); Ser. No. 16/100,351, filed Aug. 10, 2018 (10 Aug. 2018); Ser. No. 16/024,038, filed Jun. 29, 2018 (29 Jun. 2018); Ser. No. 16/013,707, filed Jun. 20, 2018 (20 Jun. 2018); Ser. No. 16/013,491, filed Jun. 20, 2018 (20 Jun. 2018); Ser. No. 15/998,041, filed Jun. 18, 2018 (18 Jun. 2018); Ser. No. 15/932,911, filed May 18, 2018 (18 May 2018); Ser. No. 15/932,906, filed May 18, 2018 (18 May 2018); Ser. No. 15/932,110, filed Feb. 5, 2018 (5 Feb. 2018); Ser. No. 15/732,955, filed Jan. 16, 2018 (16 Jan. 2018); Ser. No. 15/732,953, filed Jan. 16, 2018 (16 Jan. 2018); Ser. No. 15/732,397, filed Nov. 6, 2017 (6 Nov. 2017); Ser. No. 15/732,130, filed Sep. 20, 2017 (20 Sep. 2017); Ser. No. 15/731,804, filed Aug. 3, 2017 (3 Aug. 2017); Ser. No. 15/731,478, filed Jun. 16, 2017 (16 Jun. 2017); Ser. No. 15/482,436, filed Apr. 7, 2017 (7 Apr. 2017); Ser. No. 15/258,877, filed Sep. 7, 2016 (7 Sep. 2016); and further to the following U.S. provisional appl. Ser. No. 62/707,588, filed Nov. 8, 2017 (8 Nov. 2017); 62/600,134, filed Nov. 7, 2017 (7 Nov. 2017); and further to the following PCT appl. nos.: PCT/US17/30889, filed May 3, 2017 (3 May 2017); and PCT/US17/30448, filed May 1, 2017 (03-MAY-2017).

### FIELD OF THE INVENTION

The described invention relates generally to endovascular devices and more particularly to a specifically shaped support catheter which provides system for embolic protection. More particularly, the described invention is directed to a device that can obviate the need for an open surgical cutdown of the common carotid artery (CCA) with a carotid stent, using a flow reversal loop system for embolic protection, while also employing a percutaneous technique and novel carotid access devices which use anatomical fulcrums and/or unique steering capabilities for added support.

### BACKGROUND OF THE INVENTION

Minimally invasive treatments are increasingly popular including intravascular catheter treatments. Said treatments may be more effective than the prior art, nonetheless, in some embodiments they require an open surgical cutdown of the carotid artery which requires more anesthetic to perform typically than percutaneous procedures, with attendant anesthetic risks. Additionally, said procedures require surgical expertise, and presents additional risks of surgical injuries and/or infection at cutdown site in neck.

The present disclosure relates generally to medical methods and devices. More particularly, the present disclosure relates to methods and systems for accessing the carotid arterial vasculature and establishing retrograde blood flow during performance of carotid artery stenting and other procedures.

Carotid artery disease commonly results in deposits of plaque which narrow the junction between the common carotid artery CCA and the internal carotid artery ICA, an artery which provides blood flow to the brain. Said deposits may result in embolic particles being generated and entering the cerebral vasculature, leading to neurologic consequences such as transient ischemic attacks TIA, ischemic stroke, or death.

Various therapies exist to ameliorate carotid artery disease related difficulties. The most common are carotid endarterectomy CEA and a carotid artery stenting CAS. Both expose patients to the risk of emboli being released into the cerebral vasculature via the internal carotid artery ICA.

In response, recent prior art discloses a variety of devices and associated methods. For example, consider methods which comprises trans-cervical access and blocking of blood flow through the common carotid artery while shifting blood from the internal carotid (see U.S. Ser. No. 12/835,660, U.S. Ser. Nos. 10/996,301, 12/366,287, 12/366,287 and 15/044,493).

The prior art discloses trans-carotid arterial revascularization. In particular, a small incision is made just above the collar bone and surgical dissection is used to surgically expose the common carotid artery. A soft, flexible sheath is placed directly into the carotid artery, and a clamp is applied to the external surface of the common carotid artery around the sheath, and the sheath connected to a system that will reverse the flow of blood away from the brain to protect against fragments of plaque that may come loose during the procedure. The blood is filtered and returned through a second sheath placed in the femoral vein in the patient's thigh or another vessel. Thereupon, the prior art also discloses balloon angioplasty and stenting performed while blood flow is reversed, and after the stent is placed successfully to stabilize the plaque in the carotid artery, the clamp is released and flow reversal is turned off and blood flow to the brain resumes in its normal direction. However, the prior art requires a surgical cut-down and dissection of the common carotid artery in the neck. Said surgery tends to disfigure the patient, requires additional anesthesia, additional training, and has a risk of damaging nerves.

The prior art discloses the need for direct surgical access because of difficulties encountered with endovascular access, which can make adequate access difficult and higher risk in many cases.

Thus, there is a need for a less invasive percutaneous procedure, with lower risks of disfigurement, a lower risk of nerve injury, less use of anesthesia, and a simpler system requiring less training, thereby improving access to such treatments. The present invention addresses these unmet needs in the prior art.

The prior art discloses a set of Walzman arch fulcrum catheters, for example U.S. patent application Ser. Nos. 15/932,775 and 16/290,923, which may be useful to overcome this difficulty. In particular, a version with a balloon on distal end may be used. Using this device, a user may consistently obtain transfemoral carotid access with adequate support with little difficulties and lower risks, and achieve similar results via a percutaneous transfemoral access, with less needs for anesthetics and their attendant risks.

Additionally, the prior art discloses a set of Walzman radial access catheters, which can make safe percutaneous access of either carotid artery feasible in the vast majority of patients, for example Ser. No. 16/501,591. Said catheters may also reduce access-site complications further.). Said catheters can occasionally herein be further modified with at least one additional lumen substantially in the wall of said catheter, that can exit the wall of said catheter via at least one perforation in the outer wall of said catheter, to provide irrigation proximal to the balloon when said balloon is inflated, so as to minimize formation of clot proximal to said balloon. Such clots can form when a balloon occludes a vessel and causes stasis of blood.

### SUMMARY OF THE INVENTION

The present invention combines minimally invasive percutaneous endovascular carotid-artery access with rigorous blood flow-reversal, in order to protect the brain from embolic debris when introducing interventional devices into the carotid artery. In particular, the present invention uses reverse flow elements to prevent flow of blood to the brain, thus allowing maximal medical devices to be delivered to target areas more safely. Said reverse flow techniques, vaso-plugs, pumps, and irrigation distal to a lesion, among others.

The invention may be embodied in the form of either of two preferred devices—one for right carotid stenosis and one for left carotid stenosis. The current invention includes a balloon mounted access sheath that is optimized for percutaneous access of the right and left carotid arteries in which a portion of said balloon mounted sheath is optimized to rest upon the lesser curvature of the aortic arch, in order to increase support for subsequent delivery of balloons and stents through said sheath, while also preventing recoil and kickback of said sheath and devices into the aortic arch, thereby improving procedural efficacy and reducing procedural risks.

The current invention, in other embodiments, may use transfemoral percutaneous endovascular access via additional arch fulcrum access catheters cross-referenced hereinabove, and additionally may use any of the previously described radial access catheters described herein. Additional embodiments may use alternatively various catheters described in the prior art, along with novel devices and methods to increase flow reversal at the lesion site, while minimizing the necessary diameter of the delivery catheter, and while minimizing any potential sump effect from the brain.

In still another embodiment of the current invention, a hollow wire is employed. The advantages of using a hollow wire include the ability to infuse fluids through it. This can

A. De-rease sump effect during flow reversal to minimize distal tissue ischemia;

B. Infuse blood if there is tissue ischemia;

C. infuse neuroprotective solutions, such as cooled fluids; and

D. Infuse other material.

The benefits of using pressurized and/or pumped infusions is the ability to deliver higher rates (volume/time) of fluid through a small diameter lumen, thereby keeping the diameter of the devices as small as possible-this decreases risks at treatment site and access site, and increases range if access sites available (especially making treatment via radial artery access in most patients).

Additionally, using these methods and devices for carotid access can improve ease of percutaneous carotid access in many of the most difficult anatomical scenarios, thereby decreasing the risks of these percutaneous approaches. The sheaths optimized for right carotid access via a transfemoral route will typically have a longer segment resting on the lesser arch of the aorta than the corresponding sheath for left carotid access. Embodiments include transfemoral and arm access arch fulcrum catheters. As previously described in incorporated submissions, all catheters may optionally have active steerability of their respective bends. As is known in the art, this may sometimes be accomplished by the presence of wires in the wall of the catheter, with a mechanism to shorten the effective length of a wire to create a bend.

### BRIEF DESCRIPTION OF THE DRAWINGS

FIG. 1 shows an illustration of a cross-section view of one embodiment of tube 1 of the described invention disposed in a bovine, Type III 7000 aortic arch, having the second segment resting on the arch 2000 fulcrum with a third bend 30 and fourth segment 400 deployed in left common carotid artery 5000 with a bovine origin. Also identified are descending aortic artery 1000, right subclavian artery 3000, right vertebral artery 3500, right carotid artery 4000, innominate (brachiocephalic) artery 6000, Type Ill arch 7000, left subclavian artery 8000, and left vertebral artery 8500.

FIG. 2 shows an illustration of a cross-sectional view of one embodiment of tube 1 of the described invention in place having the second segment 200 resting on the fulcrum of arch 2000 with an obtuse (inner) third bend 30 and fourth segment 400 deployed in right subclavian artery 3000.

FIG. 3 shows an illustration of a cross-sectional view of one embodiment of tube 1 of the described invention in place having the second segment 200 resting on the fulcrum of arch 2000 with a second bend 20, and third segment 300 deployed in the right carotid artery 4000.

FIG. 4 shows a cross-sectional view of an exemplary embodiment of tube 1 having a final element 400 comprising multiple bends and segments deployed in the right subclavian artery 3000.

FIG. 5 shows an illustration of a cross-sectional view of one embodiment of the described invention in place, having tube 1 having at least one lumen, which is the interior of tube 1, said tube 1 having a first segment 100 entering from an arm vessel or axillary artery or vein; second segment 200 disposed between first bend 10 and second bend 20, resting on the fulcrum of arch 2000 and third segment 300 deployed in the left common carotid artery 5000.

FIG. 6 shows an illustration of a cross-sectional view of one embodiment of tube 1 of the described invention in place having tube 1 having at least one lumen, which is the interior of tube 1, said tube 1 having a first segment 100 entering from an arm vessel or axillary artery or vein through left subclavian artery 8000, optional first side hole 170 proximal to the entry point of left vertebral artery 8500, second segment 200 resting on aortic arch 2000, second segment including optional side hole 270, second bend 20 directing third segment 300 upward into innominate (brachiocephalic) artery 6000 wherein end hole 405 is disposed. In this embodiment, second segment 200 includes optional side hole 270.

FIG. 7 shows an illustration of a cross-sectional view of the embodiment of FIG. 5 of the described invention, in place further including at least two additional side holes 170 and 171 disposed within right subclavian artery 3000; further including optional at least one balloon element 333 proximal to end hole 405 disposed within left subclavian artery 8000, and an inflation lumen (not shown) for balloon element 333, said inflation lumen passes from the exterior of a body and extends substantially through the wall of the intraluminal portion of tube 1 to balloon element 333.

FIG. 8 illustrates the angle ranges for bend 10 (i.e., angle range 190 degrees to 280 degrees) and the angle ranges for bend 20 (i.e. angle range 70 degrees to 150 degrees), wherein the opposite angle range for bend 10 is 1111 and the opposite angle range for bend 20 is 2111. The arrow in FIG. 8 denotes direction of passage of devices from outside the body direction relative to the angles 1111 and 2111. It should be noted that bend numbers 10 and 20 have corresponding, opposing angle ranges such as 1111 and 2111, respectively. This nomenclature distinction has been to insure clarity of disclosure.

FIG. 9 illustrates an arm access arch fulcrum support catheter with three bends. In addition to bend 10 and bend 20 a third bend 30 is disclosed. Beyond bend 30 is segment 400.

FIG. 10 shows an illustration of the preferred embodiment for transfemoral percutaneous treatment of the right carotid in most anatomies, with the balloon 333 deflated. Said balloon 333 is disposed at the distal tip 405 of segment 300. More particularly, first internal curve 3111 of first bend 10 has a curvature of 70 to 120 degrees; second internal curve 4111 of second bend 20 has a curvature of 65 to 130 degrees. To reiterate, it should be noted that bend numbers 10 and 20 have corresponding, opposing angle ranges or internal curves such as 3111 and 4111, respectively. This nomenclature distinction has been to insure clarity of disclosure.

FIG. 11 shows an illustration of a preferred embodiment for the left carotid artery with the balloon 333 inflated. In this embodiment, internal curve 5111 of bend 10 has a curvature of between 60 and 120 degrees.

FIG. 12 shows an illustration of external elements of the present invention, including filter 9222, Y-connector 9223, external termination device of first catheter such as a Luer Lock element 9224, venous sheath 9225, flow regulator 9226, and stopcock 9227.

FIG. 13 illustrates an irrigation catheter 9300, having a distal end 9333, and a plurality of fluid-delivery ports 9334 (depicted in position in FIG. 18).

FIG. 14 depicts an elongated embodiment of the irrigation catheter 9300 of FIG. 13, further including angioplasty balloon element 9555, occlusion balloon element 9556, including the plurality of optional irrigation ports 9334, further having said optional irrigation ports 9334 disposed proximal to optionally tapered distal tip 9557.

FIG. 15 illustrates the interior of a narrowed internal carotid-artery lumen 7892, showing a balloon 333 disposed proximal to end hole 405 of tube 1 and narrowed area 7892, with blood-flow direction depicted by arrows. In particular, the narrowed lumen 7892 is restricted by cholesterolic plaque 7891 extending from carotid bulb 7890.

FIG. 16 illustrates the narrowed internal carotid-artery lumen 7892 of FIG. 15, additionally disclosing stent delivery catheter 8970, capable of delivering stent 8971 over solid wire 8975, further showing flow direction at the lesion by arrow.

FIG. 17 illustrates the narrowed internal carotid-artery lumen 7892 of FIG. 15, additionally disclosing an angioplasty occlusion balloon 8973, disposed upon fluid delivery catheter/hypotube 8974, blood flow direction shown by arrows.

FIG. 18 illustrates the narrowed internal carotid-artery lumen 7892 of FIG. 15, further depicting delivery wire 8972, which is shown guiding irrigation catheter 9300 having optional, multiple fluid-delivery ports 9334 (one shown in FIG. 13) disposed thereon both proximally to temporary occlusion balloon element 8975, and distally to distally delivered deflated angioplasty balloon 8973, balloon elements 8973 and 8975 being mounted on double-balloon irrigation catheter 9300. Stent 8971 is illustrated being unsheathed from stent delivery catheter 8970. Delivery catheter 8970 having a sufficient diameter to be able to be advanced over catheter/hypotube.

### DETAILED DESCRIPTION OF THE INVENTION

The term “recoil and displacement”, as used herein refers to the phenomenon of catheter prolapse or displacement (slipping forward, back, or down, and out of the desired position) due to a counterforce against the catheter by the advancing wire, second catheter, or other, additional device.

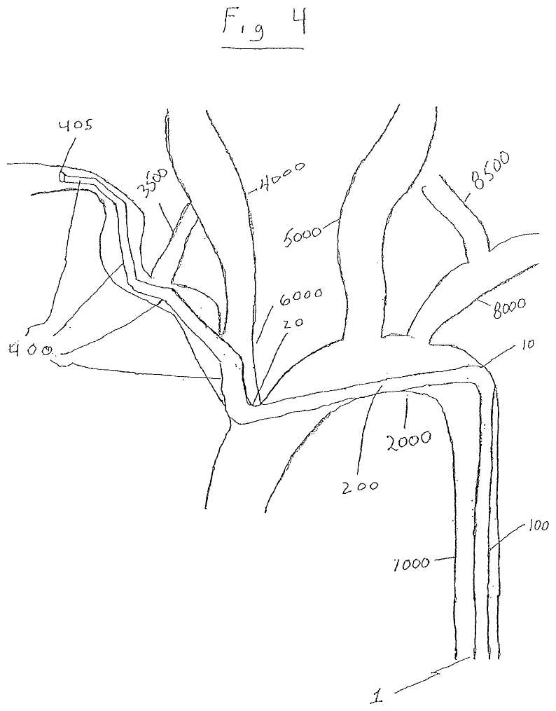

The current invention's novelty, in some embodiments, rests upon the use of an anatomical fulcrum as an anti-kickback, anti-displacement support structure. Beyond the shaping of the invention to allow said support, the invention in some embodiments deploys a final element at the distal end to facilitate delivery of said distal end to the target area. The final element of the simplest embodiment of the invention is shown in FIG. 3.

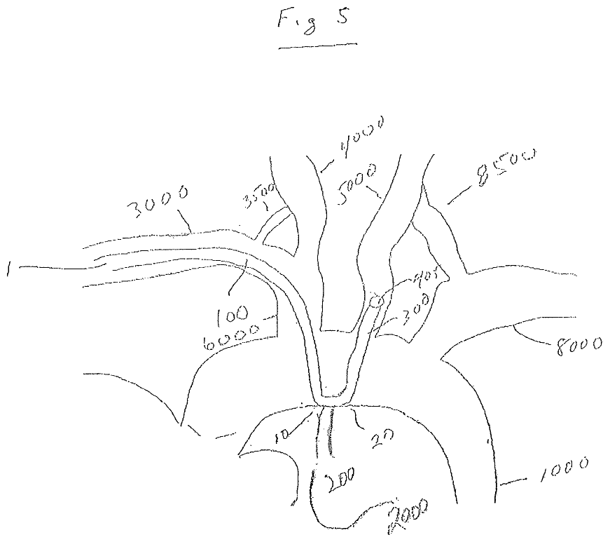

The final element in the preferred embodiment comprises two bends, three segments and one end hole. As claimed, the final element may comprise one or more additional bends and one or more additional segments beyond those comprising the preferred embodiment. The final-element configuration is determined by the path the user of the invention determines is necessary to deliver the distal end hole 405 of the current invention to the target area as illustrated in FIG. 4. Additionally, although most commonly the second segment will rest on a vascular fulcrum, any segment, or in some cases multiple segments, may utilize a vascular anatomical structure for securement, to prevent unwanted kickback and displacement. Additionally, the preferred embodiment for the current invention will have an additional circumferential balloon near its distal end hole, with at least one additional lumen to inflate and deflate said balloon positioned substantially within the wall of said catheter, in its intraluminal segment. Not pictured is a proximal branching of said additional lumen to its own proximal end hole at its own external termination device, said branching which occurs outside the patient's body, which is well known in the prior art. Inflation of said balloon is capable of completely occluding the ipsilateral common carotid artery proximal to the target stenosis, which is typically in the internal carotid artery, thereby allowing reversal of flow in said internal carotid artery when flow is allowed through the proximal sheath. This flow can be active or passive. It can be aided in some cases by proximal aspiration, vacuum, or other pump mechanism. It can be aided in some cases by differential pressure between the target artery and a vein to which a circuit of flow is subsequently established. Said vein can be accessed with a separate catheter, and tubing can connect the proximal end of said sheath with the proximal end of said separate catheter. In some embodiments a regulator along said tubing can modulate flow rates as well. In some embodiments a filter along said tubing can filter the blood and remove embolic debris before returning it to the patient as well. In some embodiments flow reversal can also be augmented by infusion of fluids across and/or distal to said target lesion, as previously described with similar devices by Walzman. Such infusions can also act to decrease a known sump effect from the brain while flow is reversed across the lesion, thereby decreasing the potential for ischemia of the brain tissue that can result from said sump effect.

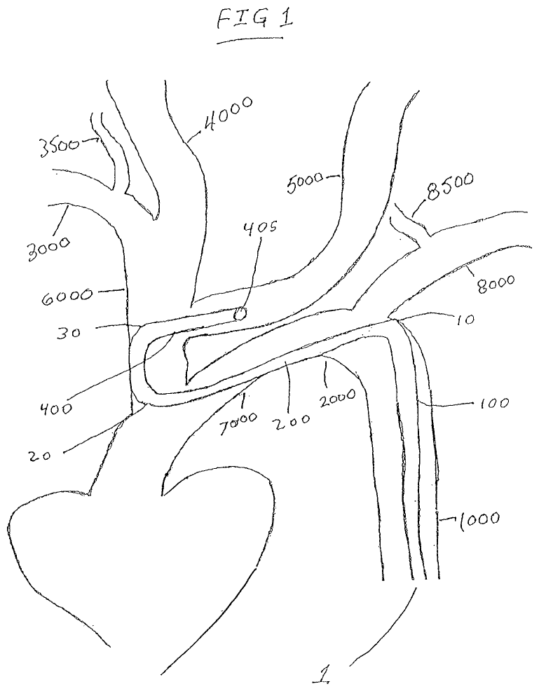

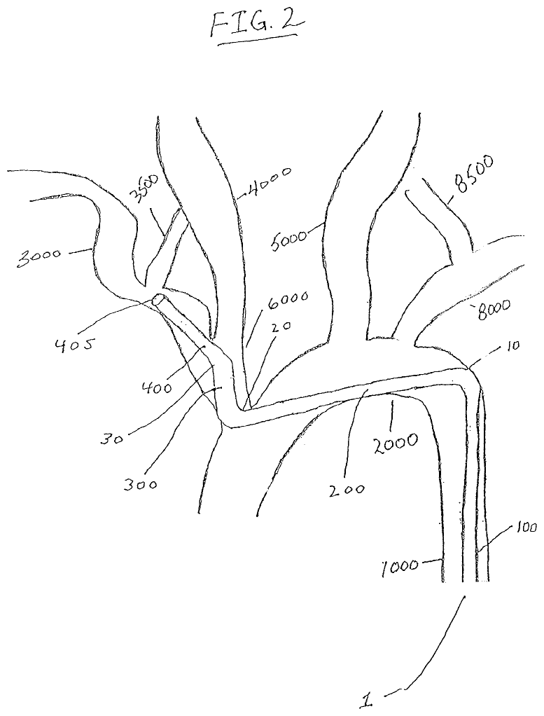

Now referring to FIG. 1, the tube of the present invention is shown deployed in the aorta with distal end hole 405 terminating in an abnormal anatomical variation of the left carotid artery 500 referred to as a bovine arch. The device of this embodiment the current invention has seven principal elements. The first three of said elements are bends, and four are segments of the tube. More particularly, first bend 10 connects segment one 100 to segment two 200 at a non-obtuse angle, as measured as an angle from the proximal catheter tubing to the tubing of the second segment, in this example.

First bend 10 may be active or passive. A passive bend, as disclosed by the prior art, is a bend which is formed by the use of a wire or a braid. A passive bend 10 has been treated in such a way prior to the introduction to the body that, if there are no other forces action, it will form a non-obtuse angle. In order to deploy a tube must be straight, so there must be a force to straighten bend 10, such as a wire, a stiff inner or outer tube or combination, such that upon removal of said external force a non-obtuse angle is formed. In other embodiments, any bend may be active or passive. In some embodiments, all bends are active; in other embodiments all bends are passive; in yet other embodiments, bends may be a mix of active bends and passive bends.

Other embodiments are adapted to access aortic arch 2000 through a vessel in the arm or, for example, from a radial artery, brachial artery, axillary artery (or vein) (not shown), when such access may be preferred. In an embodiment depicted in FIG. 5, tube 1 includes at least one lumen, a first segment 100 accessing aortic arch 2000 through right subclavian artery 3000. FIG. 5 further illustrates tube 1 having second segment 200 disposed between first bend 10 and second bend 20, resting on the fulcrum of arch 2000 and third segment 300 deployed in the common carotid artery 5000; in this embodiment, second segment 200 includes optional side hole 270.

In a variant embodiment of FIG. 5, segment 100 of tube 1 is shown as having a “gentle curve” nearest to an external termination device (not shown), and at least two bends 10 and 20.

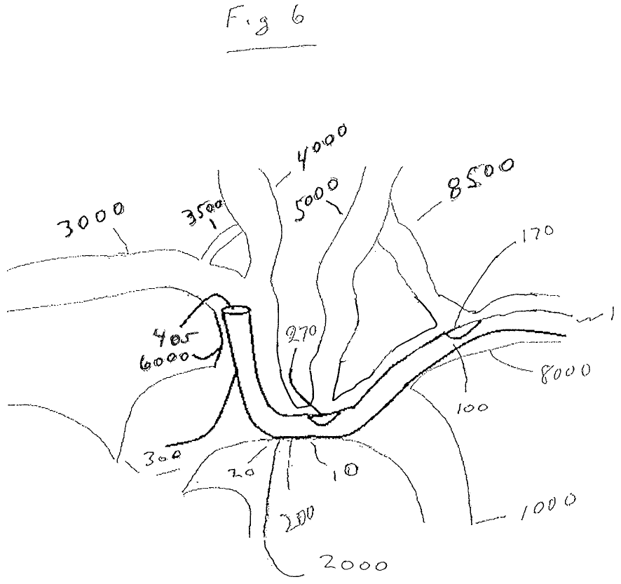

FIG. 6 illustrates tube 1 of the described invention in place in the body, wherein second segment 200 rests on aortic fulcrum 2000. Aortic fulcrum 2000. is accessed via an arm vessel or axillary artery or vein from the opposite arm illustrated in FIG. 5. In the embodiment of FIG. 6, tube 1 passes through left subclavian artery 8000. In the illustrated embodiment optional at least one side hole 170 is disposed proximal to left vertebral artery 8500. Second segment 200 rests on aortic fulcrum 2000. Second segment 200 further includes second optional side hole 270 disposed proximal to aortic fulcrum 2000. Second bend 20 directs third segment 300 upward into innominate artery 6000 wherein end hole 405 is disposed. The combination of a catheter that utilizes the inferior curve of the aortic arch as a vascular fulcrum with optional side holes, through which additional catheters can be passed, may further facilitate catheterization of bilateral vertebral and carotid arteries via a single access sight in either arm.

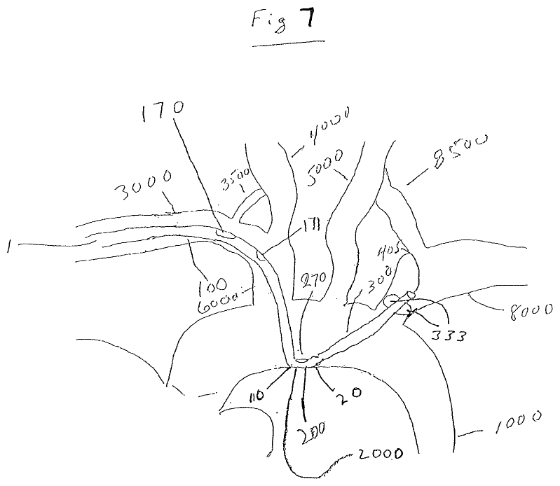

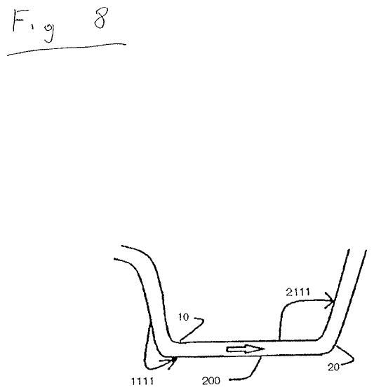

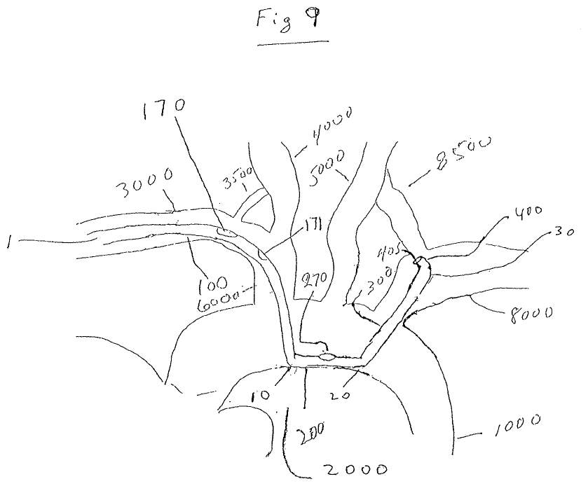

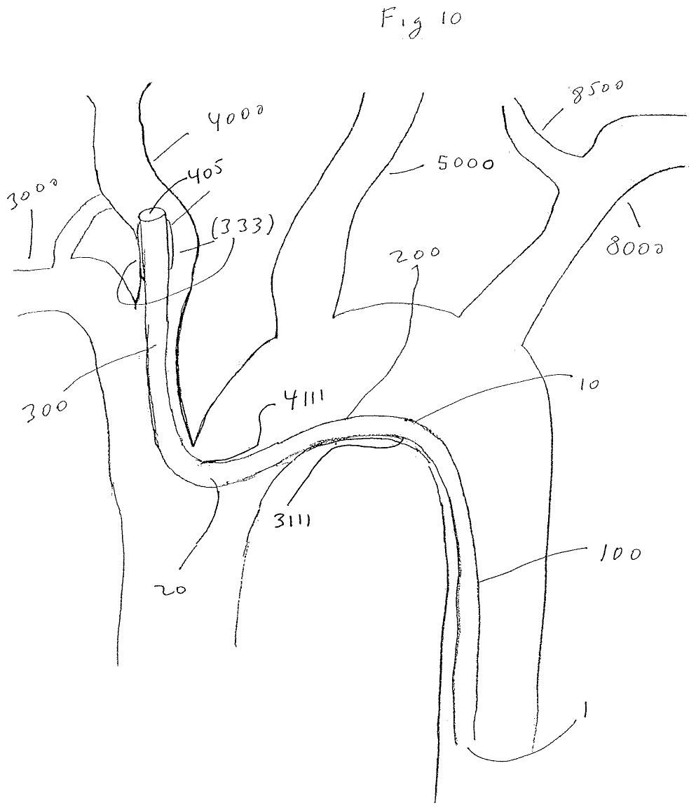

As depicted in FIG. 7, another embodiment also accesses aortic arch 2000 through a vessel an arm. In the embodiment of FIG. 7, tube 1 includes at least one lumen, a first segment 100 accesses aortic arch 2000 through right subclavian artery 3000. FIG. 7 further illustrates tube 1 having at least one optional side hole 270 disposed upon second segment 200 within aortic arch 2000, and a second optional side hole 170 disposed upon first segment 100 deployed within right subclavian artery 3000 proximal to the entry point of right vertebral artery 3500, and a third optional side hole 171 proximal to the origin of the right common carotid artery.

It should be noted that in all embodiments containing more than one balloon, each balloon may optionally require a separate inflation lumen.

Other variants of catheter embodiments optionally include at least one valve (not shown). Still other variants of catheter embodiments may optionally include at least one supplemental irrigation lumen substantially in the wall of the catheter, which may have their respective end hole(s) terminate either inside or outside the catheter, to help minimize clot formation in the exit region.

In an alternative embodiment, materials or techniques may be employed such that a non-obtuse bend is achieved. Embodiments would include shape-memory metals or polymers. In addition or in the alternative, radiation may be focused on a point of tube 1 such that bend 10 is forced to adopt a desired, non-obtuse angle of segment two relative to the proximal catheter of segment one to position segment two 200 over the fulcrum of aortic arch 2000.

Segment one 100 has a length of at least 20 cm in length and an internal diameter of from 0.1 French to 30 French. In a preferred embodiment deployed transfemorally for access of the innominate arteries distal branches with a Type II and Type III arch, first bend 10 is deployed in the artery at a non-obtuse angle to position segment two 200 for optimal positioning on the fulcrum of aortic arch 200.

Segment two 200 measures at least 3 cm in length and no more than 35 cm in length in the preferred embodiment of FIG. 3. Segment two 200 has an internal diameter of from 0.1 French to 30 French. Segment two 200 has a first end which terminated in first bend 10 and a second end which terminates in second bend 20. Second bend 20 has an angle of 90 degrees plus or minus 60 degrees relative to the catheter of segment two.

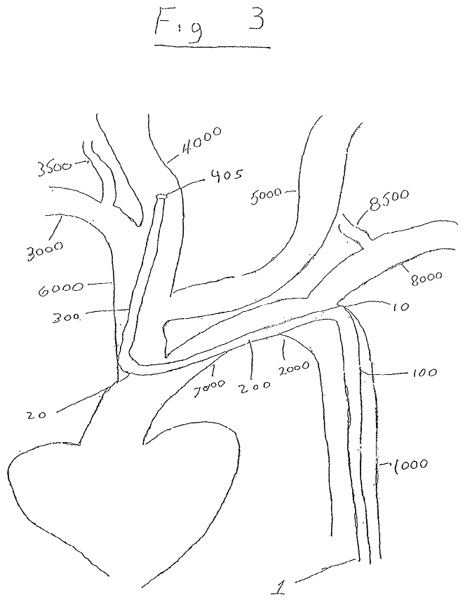

Second bend 20 connects to segment three 300 of tube 1. Segment three 300 measures at least 0.5 cm in length and has an internal diameter of from 0.1 French to 30 French. Segment three 300 has a first end which terminates in second bend 20 and connected to segment two 200 of tube 1, and a second end terminating at third bend 30. Third bend 30 (FIG. 2) has an angle of 90 degrees plus or minus 60 degrees.

Third bend 30 connects to segment four 400 (FIG. 4) of tube 1. Segment four 400 measures at least 0.5 cm in length and has an internal diameter of from 0.1 French to 30 French. Segment four 400 has a first end which terminates in third bend 30 and connected to segment three 300 of tube 1, and a second end terminating at distal hole 405.

Now referring to FIG. 2, the present invention is shown deployed in a Type III aortic arch anatomy. Segment one 100 is deployed downwardly in the ascending aorta 1000, which is located below the fulcrum formed by the arch of the aorta 2000. The middle of segment two 200 is shown resting on the fulcrum formed by the arch of the aorta 2000. Segment three 300 is shown in this example being upwardly deployed into innominate artery 6000, and segment four 400 extending upwardly from third bend 30 at an obtuse angle, relative to the catheter of segment three 300, and deployed distally in right subclavian artery 3000.

Now referring to FIG. 3, the present invention is shown deployed in a sample aortic arch anatomy. Segment one 100 is deployed downwardly in the ascending aorta 1000, which is located below the fulcrum formed by the arch of the aorta 2000. The middle of segment two 200 is shown resting on the fulcrum formed by the arch of the aorta 2000. In this embodiment the first bend 10 must be non-obtuse. While this is a feature of this embodiment, a non-obtuse bend is not a limitation of the present invention, rather a shape limitation of a particular embodiment which will allow the use of the arch of the aorta 2000 to prevent kick-back and displacement in particular anatomical scenarios. An obtuse bend will not allow the use of the arch of the aorta 2000 to prevent kick-back and displacement while obtaining transfemoral access to the right carotid artery in these select anatomical variants.

According to one embodiment, the middle segment two 200 has ridges to promote stability at the focal point 2000. According to another embodiment, the middle segment two 200 is coated with an elastic material to deform atop the fulcrum point 2000 for improved securement.

The various components of the described invention may be comprised of one or more materials. Thermoplastics include, but are not limited to, nylon, polyethylene terephthalate (PET), urethane, polyethylene, polyvinyl chloride (PVC) and polyether ether ketone (PEEK).

Thermosets include, but are not limited to, silicone, polytetrafluoroethylene (PTFB) and polyimide. Composites include, but are not limited to, liquid crystal polymers (LCP). LCPs are partially crystalline aromatic polyesters based on p-hydroxybenzoic acid and related monomers. LCPs are highly ordered structures when in the liquid phase, but the degree of order is less than that of a regular solid crystal. LCPs can be substituted for such materials as ceramics, metals, composites and other plastics due to their strength at extreme temperatures and resistance to chemicals, weathering, radiation and heat. Non-limiting examples of LCPs include wholly or partially aromatic polyesters or co-polyesters such as XYDAR® (Amoco) or VECTRA® (Hoechst Celanese).

According to some embodiments, the bends comprise a shape memory polymer (SMP). Shape memory polymers include, but are not limited to meth-acrylates, polyurethanes, blends of polystyrene and polyurethane, and PVC. According to some embodiments, the bends of the catheter comprises a shape memory alloy (SMA). Non-limiting examples of shape memory alloys include nickel-titanium (i.e., nitinol).

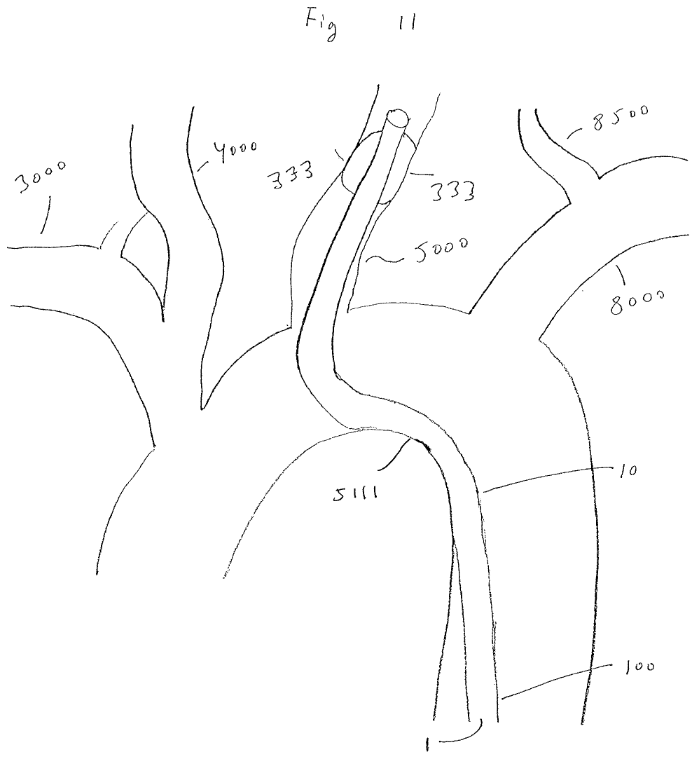

Now referring to FIG. 10 which discloses a preferred embodiment for the right carotid. The first segment 100 extends from an external termination device through a first curve 3111. In the current embodiment the distal region of the first segment, at the distal first curve, and extending into the proximal second curve 4111, which extends into the second segment 200 and curves in a substantially opposite direction, are optimized to rest upon the lesser curve of the aortic arch, thereby providing support, and preventing kickback and prolapse of the distal access sheath as well as additional medical devices that are subsequently passed through said sheath into the distal vasculature. It should be noted that second segment 200 is bounded proximally by first bend 10 and first curve 3111, and distally by second bend 20 and second curve 4111.

Said sheath has a first segment with an effective length (segment within the body) of 30 cm-70 cm, and a second segment with a length of 4 cm-25 cm when used transfemorally for carotid bifurcation pathology. The OD may be 4Fr-12Fr for this application. The sheath additionally has at least one circumferential balloon near its distal end hole, which is optimized for atraumatic temporary occlusion of the common carotid artery during angioplasty and stenting, in order to create flow reversal across the lesion. The sheath has a primary working lumen for delivery of additional medical devices such as balloons, wires, stents, etc., as well as at least one additional lumen, substantially within the wall of the effective length of said catheter, which serves exclusively to inflate and deflate said at least one circumferential balloon. The first curve, measured as the shortest angle between the proximal catheter 100 and the center of the second segment 200, is 60-120 degrees, and the second curve, measured as the shortest angle between the proximal catheter 200 and the center of the third segment 300 is 65-130 degrees, in a substantially opposite direction. A straight inner dilator is optimally used to substantially straighten said sheath during insertion into the vasculature, as is well known in the prior art/field.

Like the prior art described above, the current invention relies on flow reversal across the lesion during angioplasty and stenting to minimize the risks of thromboembolic ischemic complications during the procedure. However, whereas the cited prior art relies on a carotid open surgical cut-down, the current invention optimally uses percutaneous techniques. The current invention additionally, in the preferred transfemoral embodiment, utilizes vascular fulcrums for support of the devices, to reduce potential complications and risks. Furthermore, as previously described by Walzman, the current device additionally optionally utilizes infusion of fluid distal to the lesion during the procedure to aid in flow reversal across the lesion, while minimizing a sump effect from the brain that can contribute to ischemic complications. In order to accomplish this, these embodiments of the current invention optimally utilize a hypotube capable of irrigation, in addition to its role as an access rail for balloons mounted on their delivery catheters as well as stents mounted on their respective delivery catheters, and/or additional balloon catheters capable of irrigation as well. In other embodiments the current invention may deploy an additional temporary balloon to occlude the vessel distally.

The current invention also optionally utilizes angioplasty balloons on novel catheters that can also irrigate, and or additional irrigation catheters. Additionally, the current invention optionally utilizes a double balloon catheter, wherein one balloon is optimized for angioplasty and at least one additional balloon is optimized for atraumatic temporary balloon occlusion of a vessel. In this way, an angioplasty balloon can be advanced over a wire, said wire optionally having an inner lumen and distal end or/and side holes for irrigation, said angioplasty can be inflated across said lesion to dilate the stenosis, and then deflated. The occlusion balloon can be proximal or distal to the angioplasty balloon; in the preferred embodiment it is proximal. To reduce the number of exchanges necessary during the procedure, each of which can increase risks, said balloon can then optionally be advanced past said lesion and not removed. Said second balloon temporary occlusion balloon can then be inflated distal to said lesion, further decreasing the potential for a sump of blood flow from the brain during the procedure.

Additional fluids can then be infused through said double balloon catheter, with egress ports optionally both proximal and optionally distal to said occlusion balloon, to aid in flow reversal across the lesion proximally, and prevent clot formation distal to the occlusion balloon during balloon occlusion. Said balloon, a conventional single angioplasty balloon, and/or said irrigation catheter 9300 (or hollow wire capable of irrigation) can further optionally have a detachable hub, which is an additional novelty of the current invention. Said optional detachable hub can have pressure-mounted design or a threaded-screw design, or others.

Threaded screw designs can include a thread on the inside of the detachable hub and a corresponding opposite thread on the outside of the proximal end of the catheter, or alternatively the thread can be on the outside of the distal side of the hub and on the inside of the proximal end of the catheter. This removeable hub (not shown) will allow these devices to be used as a rail (like a wire) to deliver additional catheters, such as an angioplasty balloon mounted catheter or a stent delivery catheter, both in an “over-the-wire” configuration and in a “rapid exchange” configuration, by allowing said additional catheters to be loaded over the proximal end of these catheters after the hub is detached. Said catheters can additionally have optional valves in order to prevent deflation of a temporary occlusion balloon during hub detachment. The hub can be re-attached to allow continuation of fluid delivery and/or balloon deflation when desired.

All described catheters and wires can have tapered or non-tapered distal ends.

Stents can be self-expanding, balloon expanded, or a hybrid.

The current invention may also optionally include a plug or balloon to occlude the external carotid artery, to further ensure flow is reversed across the stenosis in the internal carotid artery during angioplasty and stenting. Said plug or balloon may be mounted on a wire or catheter, may be detachable or non-detachable, may be retrievable or non-retrievable, may be permanent or temporary. On example of a temporary detachable plug is a biodegradable hydrogel plug, which the body can recanalize.

In an optional embodiment, the device of the present invention further comprises at least one vascular plug, capable of obstructing collateral flow from a branch such as the external carotid artery. Said plug is preferably located between at least one circumferential balloon and a vascular blockage, to further ensure flow is reversed at the obstruction during angioplasty and stenting. It should be noted that in one embodiment, a patient's body will break down the plug and restore flow in a vascular branch over a set period of time.

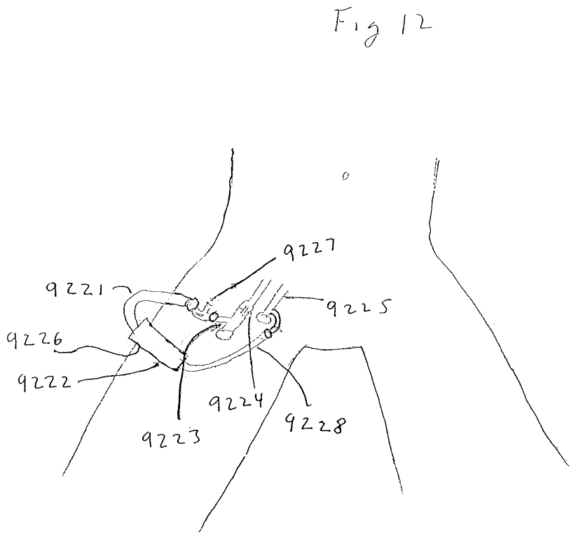

Now referring to FIG. 11 which discloses the preferred embodiment for the left carotid. The left carotid and right carotid arteries are sized in accordance with the anatomical dimensions of said arteries. Said dimensions vary from patient to patient but are readily determinable. Accordingly, embodiments intended for left carotid use will be sized as described above for the right carotid artery, with adjustments for these variations.

Now referring to FIG. 12 which illustrates the external elements of the present invention, more particularly illustrating that approach can be right femoral artery and/or left femoral artery (right illustrated). Radial, brachial, or axillary arterial access, and other percutaneous ports of access, can be used as well. Said external elements include some or all of filter 9222, Y-connector 9223, external termination device of arch-fulcrum catheter such as a Luer Lock element 9224, venous sheath 9225, flow regulator 9226, and stopcock 9227; and tube elements 9221 and 9228.

Alternatively, venous sheath 9225 can be used in any vein of sufficient size. It should also be noted that the flow regulator 9226 can be any one previously disclosed by the prior art: a wheel on a ramp (like a standard), or can involve routing blood through a higher or lower resistance path. Alternatively, the regulator can be active, utilizing pumps, artificial pressure gradients, vacuums, or other mechanisms that can increase flow through a narrow path when desired, thereby allowing a smaller sized delivery sheath to still effect flow reversal during device delivery, thereby reducing potential for access site complications, and increasing available ports of entry.

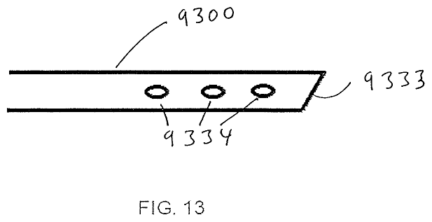

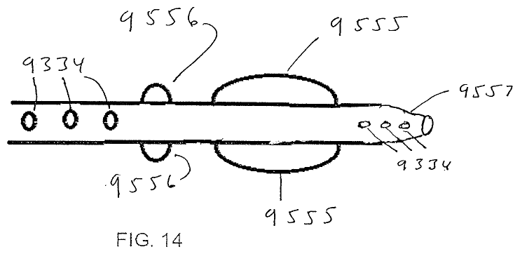

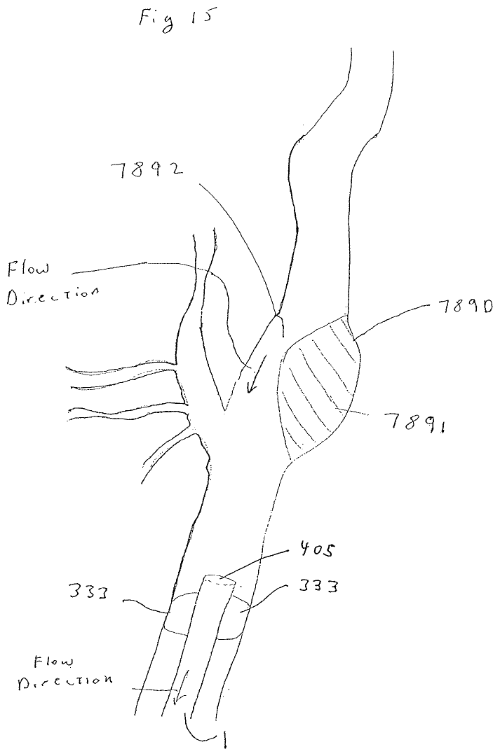

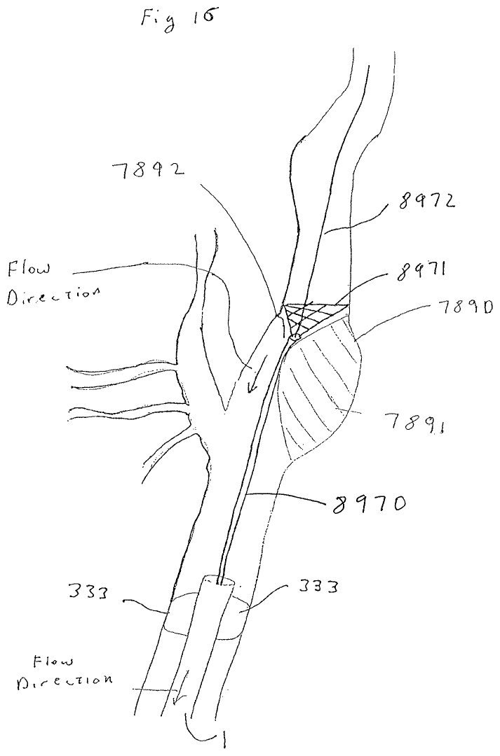

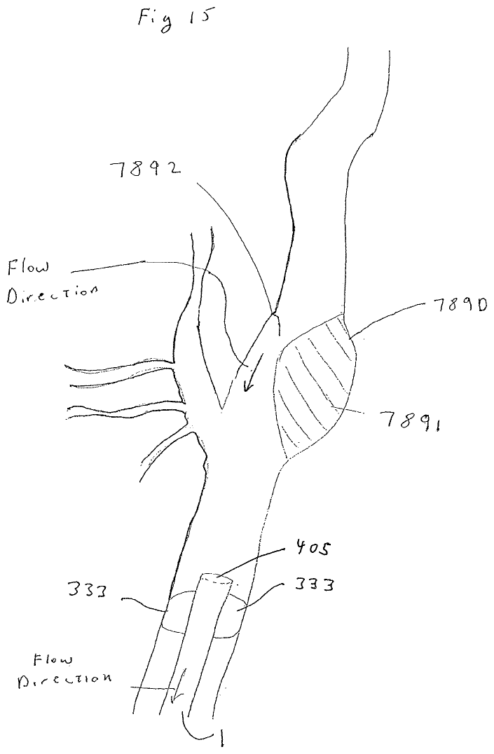

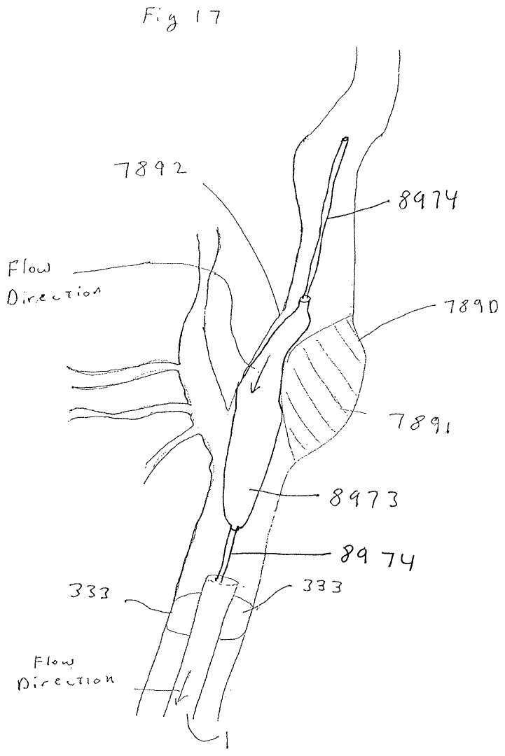

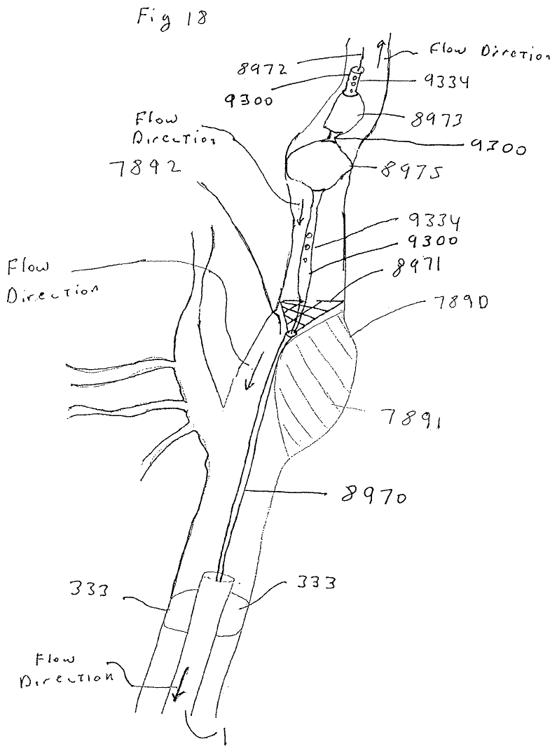

FIG. 13 illustrates one example of an irrigation wire 9300, having a distal end 9333, and a plurality of irrigation ports 9334.

FIG. 14 depicts an elongated embodiment of the irrigation wire 9300 of FIG. 13, further including angioplasty balloon element 9555, occlusion balloon element 9556, including the plurality of optional irrigation ports 9334, further having said optional irrigation ports 9334 disposed proximal to tapered distal tip 9557. A still further embodiment also optionally comprises a “peel away sheath” (not shown) to protect the access artery from the balloon, and the balloon from the access artery and tissue, during insertion of the balloon. The peel-away-sheath wall can be very thin, and the proximal sheath can optionally have a slightly larger OD to prevent leakage around it after the peel-away sheath is removed after the balloon at the distal end of the sheath is entirely intravascular.

In a still further embodiment, the disclosed device further includes at least one of series of angioplasty balloons and/or stent delivery catheters with removeable hubs and/or side ports. Said series can be delivered over each other, such that a first delivery wire “rail” crosses a lesion. Then an angioplasty balloon is inflated, with flow reversed, optionally aided by active pumps or similar. As such, the current invention can have the hub and side port of the angioplasty balloon be removable. The method simply requires that the user advance the balloon, after angioplasty inflation and subsequent deflation, slightly past a target blockage. Then the user slides the next balloon catheter, or the stent catheter, over the balloon catheter. In the prior art, systems require exchanging the balloon catheter for another larger balloon or the stent. This maneuver enhances risk to patients; for example, the wire can move, the time for the procedure be increased, and/or an increased loss of blood.

In said embodiment, the angioplasty balloon catheter is sometimes further capable of delivering fluid, which can be delivered distal to the blockage and/or across the blockage. Thereby, the any potential “sump effect” of blood flow diversion from the distal tissue is reduced, while flow is reversed across the blockage.

Referring now to FIGS. 15 through 18, it should be noted that the present invention is capable of deployment of multiple therapeutic devices into a narrowed artery lumen 7892 (FIG. 15), over delivery wire 8975, guiding irrigation hypotube 8974, and/or catheter 9300. Irrigation devices 9300 and 7892 may include optional, multiple fluid-delivery ports 9334 (shown in detail in FIG. 13). Ports 9334 may be disposed both proximally to temporary occlusion balloon element 8975, and distally to delivered angioplasty balloon 8973, both balloon elements 8973 and 8975 being mounted on irrigation catheter 9300. Stent element 8971 may also be delivered over the exterior of irrigation catheter 9300 for simultaneous deployment.

It should be further noted that the present invention implements a balloon-guide catheter (or sheath) capable of occluding the target CCA. As indicated in FIG. 18, when the device of the present invention is deployed, fluid flow distal to the temporary occlusion balloon 8975 is static or flows distally, whereas proximally to temporary occlusion balloon 8975 fluid flows proximally through stent 8971, then through tube 1. In a preferred embodiment of the present invention, delivery catheter 8970 is dimensioned sufficiently relatively smaller in diameter versus the inner diameter of tube 1 to allow proximal blood to flow through to tube 1, while having a sufficient inner diameter to deliver over irrigation device 9300.

In an alternative embodiment, the described invention relates generally to endovascular devices and more particularly to a specifically using a shaped support catheter and a hypotube in lieu of a wire to shape catheters, as disclosed by the invention. More particularly, the described invention is directed to a device which uses hypotubes, and related elements to obviate the need for open surgical cutdowns of the common carotid artery (CCA) with a carotid stent, using a flow reversal loop system for embolic protection, while also employing a percutaneous technique and novel carotid access devices which use anatomical fulcrums for added support.

Additionally with respect to the hypotube alternative embodiment, the present invention combines direct carotid-artery access with rigorous blood flow-reversal, in order to protect the brain from embolic debris when introducing interventional devices into the carotid artery. Disclosed is a medical device capable of treating vascular blockages, more particularly a hypotube having at least one lumen from a proximal port to a distal end hole, capable of delivering additional medical devices; at least one distal, circumferential balloon capable of temporary occlusion of native flow in a vessel near its distal end hole upon inflation. The disclosed hypotube is capable of delivering a second balloon for angioplasty, and at least one stent.

While other Walzman inventions have disclosed the combined use of a wire for curving tubes, and a stent delivery catheter, the present invention discloses a hypotube to perform both of these functions. This configuration eliminates at least one element, thus simplifying the invention, and reducing the possibility of failure. Additionally, by replacing the wire and delivery catheter with a hypotube, the hypotube will be smaller that the combination of those two, thus allowing access to smaller vessels.

With respect to the hypotube embodiment of the present invention the hypotube element replaces the stent delivery catheter of the initially disclosed structure. Thus, FIGS. 16, 17 and 18 illustrate the proper relationship of each element, certain elements may be interchanged. For example, when the stent delivery catheter is replaced by a hypotube, FIGS. 16, 17 and 18 the 8970 element which was a stent delivery catheter is now a hypotube. More specifically,

In this alternate embodiment, FIG. 16 illustrates the narrowed internal carotid-artery lumen 7892 of FIG. 15, additionally disclosing stent delivery hypotube 8970, capable of delivering stent 8971 and extend hypotube 8970 beyond stent 8971; further showing flow direction by arrows.

In this alternate embodiment, FIG. 17 illustrates the narrowed internal carotid-artery lumen 7892 of FIG. 15, additionally disclosing an angioplasty occlusion balloon 8973, disposed upon a fluid delivery hypotube 8970, the direction of blood flow shown by arrows.

In this alternate embodiment, FIG. 18 illustrates the narrowed internal carotid-artery lumen 7892 of FIG. 15, further depicting delivery hypotube 8970, which is shown having optional, multiple fluid-delivery ports 9334 (shown in FIG. 13) disposed thereon both proximally to temporary occlusion balloon element 8975, and distally to delivered angioplasty balloon 8973, balloon elements 8973 and 8975 being mounted on double-balloon irrigation catheter 9300; additionally, stent 8971 is also mounted on the exterior of irrigation catheter 9300. Hypotube 8970 having a diameter allowing balloon elements 8973 and 8975, and stent element 8971 to be mounted on the outer surface of hypotube 8970.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

Any publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of e present invention. All such modifications are intended to be within the scope of the claims appended hereto.

What is claimed is:

1. A medical device for treating a vascular blockage such as a narrowing comprising:
a catheter comprising a tube,
said tube comprising at least one proximal end hole and at least one distal end hole, and at least one primary lumen extending therethrough, with at least one circumferential balloon disposed near a distal end of said tube, wherein said at least one primary lumen is capable of delivering additional medical devices therethrough, and at least one additional lumen disposed within the wall of said catheter which serves to inflate and deflate said at least one circumferential balloon,
wherein said at least one balloon is capable of temporary occlusion of native flow in a vessel when inflated, and
wherein said catheter is an arch fulcrum support catheter, having
at least one wire capable of crossing said narrowing, and subsequently capable of facilitating subsequent delivery of additional medical devices over said wire, and
at least one second balloon capable of angioplasty;
at least one stent;
wherein when said at least one circumferential balloon is inflated and native flow is occluded, and flow is allowed through a proximal end port of said catheter, flow across said vascular narrowing is reversed, thereby promoting flow of any debris released during angioplasty and stenting to flow out of said catheter, avoiding potential thromboembolic complications in the distal vasculature.

2. The device according to claim 1, wherein said arch fulcrum catheter for transfemoral treatment of cervical carotid bifurcation stenosis (nonlimiting) comprises:
a first segment having at least one external termination device on its proximal end, said first segment having a useable length of approximately 35 cm-70 cm, a first curve measuring 70-120 degrees at the distal end of said first segment, said first curve having a curvature approximately 0.1 cm-15 cm length;
a second segment having a length of approximately 0.1 cm-5 cm, a second curve in a dimensionally opposite direction of 65-125 degrees, said second segment being substantially straight between said first curve and said second curve,
a third segment located distal to said second bend for delivery of therapeutic devices into distal vasculature, said third segment having a length of approximately 3 cm-20 cm, at least one circumferential balloon disposed upon the surface of said third segment, disposed proximally to an end-hole of the distal end of said third segment, such that the distal area of said first curve or said second segment abuts a lesser curvature of an aortic arch when said distal end hole is positioned in the common carotid artery near a carotid bifurcation, said vascular arch serving as a fulcrum to prevent kickback and prolapse of said catheter and said therapeutic devices.

3. The device according to claim 2, further comprising a connecting tube and a vacuum.

4. The device according to claim 2, further comprising connecting tubing, and an additional catheter for passive return of blood.

5. The device according to claim 4, further comprising a flow regulator for return of blood to a vessel.

6. The device according to claim 5, further comprising a filter and a pump for return of blood to a vessel.

7. The device according to claim 2, further comprising a peel-away sheath to facilitate insertion of said circumferential balloon during insertion into body, wherein the outer diameter of the proximal segment of said catheter is equal to or greater than the outer diameter of said peel-away sheath.

8. The device according to claim 2, further comprising at least one wire in the wall of said catheter capable of curving said tube.

9. The device according to claim 2, further comprising a peel-away sheath to facilitate insertion of said circumferential balloon during insertion into a body, wherein the outer diameter of the proximal segment of said catheter is not less than the outer diameter of said peel-away sheath, thus capable of sheathing said catheter.

10. The device according to claim 9, further comprising at least one vascular plug, capable of obstructing collateral flow from a branch between said at least one circumferential balloon and said vascular blockage, to further ensure flow is reversed at said obstruction during angioplasty and stenting.

11. The device according to claim 9, wherein said plug is permanent.

12. The device according to claim 9, wherein said plug is retrievable.

13. The device according to claim 9, wherein said plug is primarily a hydrogel.

14. The device according to claim 9, wherein said plug is degradable.

15. The device according to claim 1, further comprising at least one flow reversal device selected from the group consisting of at least one angioplasty balloon catheter having removeable hubs or side ports, and at least one stent delivery catheter, such that said at least one flow reversal device is deliverable over another said at least one flow reversal device.

16. The device according to claim 16, wherein said at least one angioplasty balloon catheter is capable of delivering a fluid distal to or across said vascular blockage.

17. The device according to claim 8, further comprising connecting tubing, and an additional catheter for passive return of blood.

18. The device according to claim 17, further comprising a flow regulator for return of blood to a vessel.

19. The device according to claim 18, further comprising a filter and a pump for return of blood to a vessel.

20. The device according to claim 2, further comprising at least one vascular plug, capable of obstructing collateral flow from a blood-vessel branch between the at least one circumferential balloon and said vascular blockage, to further ensure flow is reversed at said obstruction during the angioplasty and stenting.

21. The device according to claim 2, wherein said plug is permanent.

22. The device according to claim 2, wherein said plug is retrievable.

23. The device according to claim 2, wherein said plug is primarily a hydrogel.

24. The device according to claim 2, wherein said plug is degradable.

25. The device according to claim 1, wherein said at least one circumferential balloon is replaced with a circumferential hydrogel, said hydrogel being capable of swelling and unswelling upon exposure to fluid and an additional stimulus.

26. The device according to claim 1, further comprising a hollow wire.

27. A medical device for treating a vascular blockage such as a narrowing comprising:
a catheter comprising a tube,
said tube comprising at least one proximal end hole and at least one distal end hole, at least one primary lumen extending therethrough, with at least one circumferential balloon disposed near a distal end of said tube, wherein said at least one primary lumen is capable of delivering additional medical devices therethrough, and at least one additional lumen disposed within the wall of said catheter which serves to inflate and deflate said at least one circumferential balloon, wherein said at least one balloon is capable of temporary occlusion of native flow in a vessel when inflated, and
wherein said catheter is an arch fulcrum support catheter, having at least one hypotube capable of crossing said narrowing, and subsequently capable of facilitating subsequent delivery of additional medical devices over said at least one hypotube, and
at least one second balloon capable of angioplasty;
at least one stent;
wherein when said at least one circumferential balloon is inflated and native flow is occluded, and flow is allowed through a proximal end port of said catheter, flow across said vascular narrowing is reversed, thereby promoting flow of any debris released during angioplasty and stenting to flow out of said catheter, to avoid thromboembolic complications in distal vasculature.

28. A medical device for treating a vascular blockage such as a narrowing comprising:
a catheter comprising a tube,
said tube comprising at least one proximal end port and at least one distal end port, and at least one primary lumen extending therethrough, having at least one circumferential balloon near its distal end,
wherein said balloon is capable of temporary occlusion of native flow in a vessel when inflated circumferentially, said balloon comprising a hydrogel capable of swelling and unswelling upon exposure to fluid and an additional stimulus;
at least one hypotube capable of crossing said narrowing, and subsequently capable of serving as a rail facilitating subsequent delivery of additional medical devices over said hypotube,
wherein said hypotube is also capable of delivering fluid,
at least one second balloon capable of angioplasty,
at least one stent,
wherein when said at least one circumferential balloon is inflated, occluding native flow, said flow being allowed through said proximal end port said catheter, flow across said narrowing is reversed, thereby promoting flow of debris released during said angioplasty and stenting to flow out of said catheter to avoid thromboembolic complications in distal vasculature.
